(19) United States
(12) Patent Application Publication
(10) Pub. No.: US 2019/0122525 A1
(43) Pub. Date: Apr. 25, 2019

(54) DEVICE AND METHOD FOR MONITORING A DRIVER OF AN AUTOMOTIVE VEHICLE

(71) Applicant: VALEO COMFORT AND DRIVING ASSISTANCE, Créteil (FR)

(72) Inventor: Valérie Lancelle, Créteil (FR)

(73) Assignee: VALEO COMFORT AND DRIVING ASSISTANCE, Créteil (FR)

(21) Appl. No.: 16/082,171

(22) PCT Filed: Mar. 1, 2017
(86) PCT No.: PCT/EP2017/054833

§ 371 Date: Dec. 28, 2018

(30) Foreign Application Priority Data
Mar. 1, 2016 (FR) 1651689

(51) Int. Cl. G08B 21/06; A61B 5/18; A61B 5/00; B60Q 9/00

(52) U.S. Cl. CPC G08B 21/06; A61B 5/18; A61B 5/0077; A61B 5/746; B60Q 9/00; A61B 5/4812

(57) ABSTRACT

The invention relates to a method for monitoring the driver of an automotive vehicle comprising the following steps: a) estimation of a state of vigilance of said driver and, b) emission of at least one alarm signal intended to inform the driver regarding their state of vigilance, as a function of the driver's estimated state of vigilance, this method furthermore comprising the following prior steps of personalizing the alarm: choice by the driver of said at least one alarm signal suitable for being emitted in step b), from among a predetermined set of alarm signals, recording of each chosen alarm signal, in correspondence with an identifier of the driver in a personalized database. The invention also relates to the associated device.

### TECHNICAL FIELD OF THE INVENTION

The present invention generally relates to the field of monitoring the driver of a motor vehicle.

More specifically, it relates to a device and to a method for monitoring this driver.

### TECHNOLOGICAL BACKGROUND

It is known for the driver of a motor vehicle to be monitored in order to detect a possible loss of alertness (i.e. a loss of their ability to drive) that can result in insufficient control of the vehicle.

For example, this loss of ability can result from drowsiness and/or from the distraction of the driver.

To this end, a driver monitoring parameter is measured and interpreted by a control unit so as to deduce information therefrom that relates to the state of alertness of the driver. Where necessary, an alarm signal is emitted to notify the driver of their state of alertness.

However, the driver generally considers these alarm signals to be intrusive and disruptive. Therefore, it has often been noted that the driver ignores the alarm signals emitted by the monitoring device, and most often quickly deactivates the monitoring device.

In these conditions, the existing monitoring devices are of limited interest and do not significantly help to improve motor vehicle safety.

### SUBJECT MATTER OF THE INVENTION

In order to overcome the aforementioned disadvantage of the prior art, the present invention proposes a monitoring method and a device allowing the driver to personalize the emission of the one or more alarm signal(s) when a reduction in the alertness of the driver is detected, namely a reduction in the level of awareness and/or the level of attention of the driver.

More specifically, the invention proposes a method for monitoring the driver of a motor vehicle, comprising the following steps:

a) estimating a state of alertness of said driver; and

b) emitting at least one alarm signal intended to notify the driver of their state of alertness, in accordance with the estimated state of alertness of said driver,

and further comprising the following prior steps of personalizing the alarm signal:
selecting, by the driver, said at least one alarm signal adapted to be emitted during step b), from a predefined set of alarm signals;
recording each selected alarm signal in correspondence with a driver identifier in a personalized database.

The method for monitoring the driver according to the invention advantageously allows selection, by the driver, of the one or more preferred alarm signal(s) and the compilation of a driver file related to a driver identifier.

The method for monitoring the driver and the device according to the invention, which is personalized by the driver themselves, are better accepted and thus can completely fulfill their safety role.

Other non-limiting and advantageous features of the method according to the invention, taken individually or according to all the possible technical combinations, are as follows:
during the selection step, the driver selects each alarm signal from one or more signal(s) of a visual, audible or haptic type, or from any one of their combinations;
during the selection step, the driver also makes a selection for a predefined number of repetitions of the emission of said alarm signal to be performed as well as, optionally, a delay between two repetitions of this alarm signal and, during the recording step, the number of repetitions and the delay between two repetitions are recorded in said personalized database, in correspondence with the driver identifier;
with said estimated state of alertness belonging to a predefined set of possible states of alertness, during the selection step, the driver associates each selected alarm signal with one of said possible states of alertness of the driver and, during the recording step, the selected alarm signal is recorded in correspondence with the possible associated state of alertness;
estimating the state of alertness comprises estimating a state of drowsiness or distraction;
during step a), the state of drowsiness of the driver can be estimated as being an alert aware, loss of alertness or sleepiness state, and the state of distraction of the driver can be estimated as being a concentrated, slightly distracted or highly distracted state and wherein, during step b), said alarm signal is emitted when the state of drowsiness of the driver indicates that they have lost alertness or are in the process of falling asleep or when the state of distraction of the driver indicates that they are slightly or highly distracted;
during the selection step, the driver associates the selected alarm signal with each possible state of distraction or drowsiness and, during the recording step, said selected alarm signal is recorded in the personalized database in correspondence with the associated possible state of distraction or drowsiness;
the identifier of the driver of the vehicle is automatically determined and, during step b), the alarm signal is emitted that is recorded in the personalized database in correspondence with this identifier and corresponding to the state of alertness of the driver estimated in step b);
the identifier of the driver of the vehicle is determined automatically, the personalized database associated with this identifier is recovered and the driver is asked to modify, where necessary, each selected alarm signal for the current trip;
prior to the selection step, the driver tests each alarm signal; and
a prior step is carried out of testing the effectiveness of each alarm signal adapted to be emitted to notify the driver of their state of alertness.

The invention also relates to a device for monitoring the driver of a motor vehicle, comprising:
a device for estimating a state of alertness of the driver;
a device for emitting an alarm signal intended to notify the driver of their state of alertness, in accordance with the estimated state of alertness of the driver;
an interactive interface with the driver allowing them to select one or more alarm signal(s) adapted to be emitted by the emission device, from a predefined set of alarm signals;
a personalized database, in which each selected alarm signal is recorded in correspondence with a driver identifier.

### DETAILED DESCRIPTION OF AN EMBODIMENT

The following description, which is provided with reference to the accompanying drawings, which are provided by way of non-limiting examples, will provide an understanding of the nature of the invention and of how it can be produced.

In the accompanying drawings:

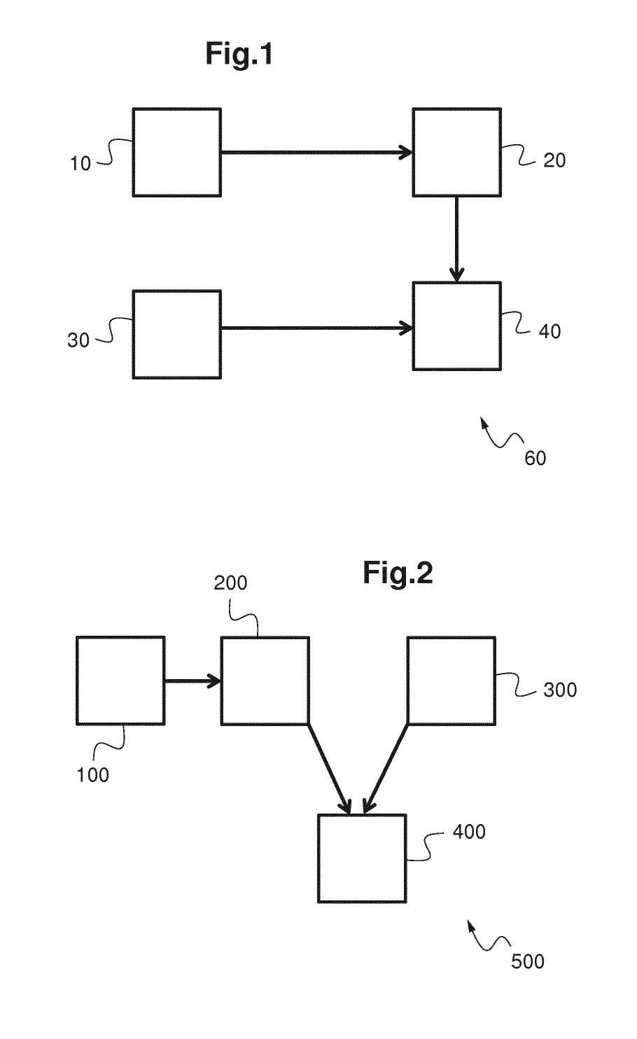

FIG. 1 schematically shows the various steps of the monitoring method according to the invention;

FIG. 2 schematically shows the various elements of the monitoring device according to the invention.

### DEVICE

FIG. 2 schematically shows the various elements of a monitoring device according to the invention.

The monitoring device 500 according to the invention comprises:
a device 300 capable of estimating a state of alertness of the driver;
a device for emitting an alarm signal 400 in accordance with the estimated state of alertness of the driver and intended to notify the driver of their state of alertness;
an interactive interface 100 with the driver allowing them to select one or more alarm signal(s) adapted to be emitted by the emission device, from a predefined set of alarm signals; and
a personalized database 200, in which each selected alarm signal is recorded in correspondence with a driver identifier.

The device 300 capable of estimating the state of alertness of the driver advantageously allows various states of alertness of the driver to be identified.

The device capable of estimating the state of alertness of the driver can estimate at least two states of alertness, comprising at least one satisfactory state of alertness and one unsatisfactory state of alertness.

Preferably, the device capable of estimating the state of alertness of the driver estimates a state of alertness belonging to a predefined set of possible states of alertness.

Advantageously, the device 300 capable of estimating the state of alertness of the driver comprises a device for measuring a driver monitoring parameter and a control unit, which receives information from this measuring device and is programmed to estimate the state of alertness of the driver in accordance with the received information.

Preferably, the measuring device records driver related information without interacting with them, thus without disrupting them. In particular, the measuring device does not emit any signal toward the driver. This has the advantage of allowing the measuring device to record the natural behavior of the driver, without any disruption or restriction by the measuring device.

The device for measuring the monitoring parameter of the driver thus does not interact with the driver.

By way of a variation, a measuring device that interacts with the driver may be contemplated, which device emits, for example, a signal intended for the driver and detects their response.

In the embodiment described herein, the measuring device comprises at least one device for capturing an image of the head of the driver.

This image capturing device comprises, for example, a camera disposed opposite the driver, for example, on the dashboard of the vehicle, in the vicinity of the speed and/or rpm dials of the vehicle.

By way of a variation, it can comprise any image capturing device that is positioned so that the face of the driver, in their driving position, enters the field of this image capturing device.

For example, said monitoring parameter measured by the measuring device relates to the viewing direction of the driver and/or to the movements of the eyes and/or to the focusing of the gaze and/or to the position of the head of the driver and/or to the closure of the eyelids of the driver and/or to a contraction of the face muscles of the driver and/or to the movements of the lips of the driver.

This measured monitoring parameter of the driver is determined in accordance with at least one image of the head of the driver captured by the measuring device.

This parameter can also be determined on the basis of two or more images of the head of the driver.

It then can also include statistical information relating to a set of data measured on a plurality of image captures of the head of the driver.

In particular, when the monitoring parameter of the driver relates to the viewing direction of the driver, the image capturing device can comprise any device for monitoring the viewing direction that is known to a person skilled in the art. The monitoring parameter relating to the viewing direction of the driver particularly can be the viewing direction in a reference frame associated with the image capturing device or in a reference frame associated with the head of the driver, or the position of the pupils of the driver in such a reference frame. It can comprise a period and/or a frequency of the shift of the gaze of the driver from the road.

It can also comprise a period for focusing on the same point, i.e. the period during which the viewing direction is constant, a frequency of the instants of focusing on the same point, a frequency and a period of twitching of the eyes, i.e. movements of the eyes between two instants of focusing on the same point.

The monitoring parameter can also comprise statistical information relating to a set of viewing directions measured from a plurality of image captures taken during a predefined period, for example, an average direction of the gaze, a standard deviation of this average direction, a statistical distribution of the viewing directions.

The driver monitoring parameter relating to the posture of the head can comprise a position and an orientation of the head of the driver in the reference frame associated with the image capturing device. It can also comprise, for example, statistical information relating to a set of postures measured from a plurality of image captures taken during a predefined period, a frequency spectrum of the head movements, or an average position of the head in a given time interval.

The driver monitoring parameter relating to the closure of the eyelids of the driver can comprise a blinking frequency of the eyes, a closure period of the eyelids, a speed of closure of the eyelids, a blinking period of the eyes, a classification of the various types of closures of the eyes, or mean or standard deviation type statistical information relating to the closure frequency or period of the eyelids of the driver.

The monitoring parameter relating to a contraction of the face muscles typically comprises a contraction frequency of the face muscles or statistical information of the mean type or statistical distribution of the contraction frequency of the face muscles. This contraction of the face muscles indicates a yawn, for example.

The measuring device can also comprise two image capturing devices allowing a three-dimensional image of the head of the driver to be reconstituted by stereoscopy.

By way of a variation, the measuring device can comprise other types of sensors allowing other types of monitoring parameters to be measured, in addition to at least one of the previously mentioned monitoring parameters, or by replacing this monitoring parameter.

For example, the measuring device can comprise a sensor adapted to measure the hand pressure on the steering wheel of the vehicle.

The measuring device can comprise, for example, a sound sensor capable of measuring the sound volume in the passenger compartment of the vehicle.

The measuring device can also comprise, for example, one or more biometric sensor(s) allowing measurement, for example, of the heart rate and/or the respiration rate and/or the frequency and/or the amplitude of a variation in the size of the pupil of the driver and/or the body temperature of the driver and/or the blood sugar level of the driver and/or the brain activity of the driver.

The control unit 300 of the device that is capable of estimating the state of alertness of the driver is programmed to estimate a state of alertness of the driver in accordance with an estimation rule and the measured value of said monitoring parameter.

The estimation rule is implemented by an estimation module of the control unit.

As previously stated, the invention can provide at least two states of alertness corresponding to satisfactory or unsatisfactory states of alertness.

By way of an example, when the measured monitoring parameter relates to the viewing direction of the driver, the state of vigilance of the driver can be considered unsatisfactory when the viewing direction of the driver is directed away from the road for a period of more than 2 seconds or because the eyelids of the driver are closed for a period of more than 500 milliseconds.

More specifically, in the first case, it is the attention level of the driver that is insufficient, whereas in the second case the level of awareness of the driver is involved.

Advantageously, the device for estimating the state of alertness of the driver is capable of estimating the state of alertness of the driver from a predefined set of possible states of alertness.

Thus, the device for estimating the state of alertness of the driver can precisely estimate the state of alertness in accordance with a state of distraction and/or a state of drowsiness.

The estimation rule preferably comprises as output a plurality of possible distraction states and/or drowsiness states of the driver, corresponding to various possible states of alertness of the driver.

The following states of alertness can be provided for estimating the state of distraction of the driver: concentrated, slightly distracted, highly distracted.

The “concentrated” state of distraction of the driver corresponds to a satisfactory state of alertness for driving.

On the contrary, the “slightly distracted” and “highly distracted” states of distraction of the driver correspond to an unsatisfactory state of alertness for driving.

Such a situation typically occurs when the driver shifts their gaze from the road in order to consult their telephone or to look inside the passenger compartment of the vehicle, for example.

This situation corresponds to a visual distraction of the driver.

The measured monitoring parameter in this case is, for example, a parameter relating to the viewing direction of the driver.

The driver can also shift their attention from driving due to an audible distraction. Such a situation typically occurs when excessive noise is present in the passenger compartment of the vehicle, for example, due to a ring tone, music playing or a lively discussion.

The driver can also shift their attention from driving due to a cognitive distraction. Such a situation typically occurs when the driver is absorbed in a discussion, on the telephone, for example, or by their own thoughts.

These latter two situations can be detected, for example, by measuring a monitoring parameter relating to the movement of the eyes and/or to the focus of the gaze.

It is also possible to determine whether the distraction of the driver is of a visual, audible or cognitive nature.

Processing images recorded by the measuring device can provide indications relative to the nature of the distraction. It is also possible to use, for example, in addition to the image capturing device of the measuring device, a sound sensor placed inside the passenger compartment.

Advantageously, the following states of distraction therefore can be provided: low visual and/or audible and/or cognitive distraction and high visual and/or audible and/or cognitive distraction.

The following states advantageously are provided to estimate the state of drowsiness of the driver: aware alert, loss of alertness and sleepiness.

The aware alert state is considered to be a satisfactory state of alertness of the driver.

On the contrary, loss of alertness and sleepiness correspond to an unsatisfactory state of alertness of the driver.

Loss of alertness, also called the relaxed aware state, corresponds to a lowered aware level characterized by specific schemes of brain activity, particularly the presence of alpha brain waves, the frequency of which varies from 8 to 12 Hertz (Hz). Body movements are more infrequent than in the alert aware state, reaction time is extended. Attention is more labile, therefore it is more difficult to concentrate on a task.

Sleepiness corresponds to an intermediate state between sleep and awareness characterized by specific schemes of brain activity, particularly the presence of theta brain waves, the frequency of which varies from 3.5 to 7.5 Hertz (Hz), slow ocular movements and a reduction in muscular tone. The presence of yawning can be detected. If the driver is fighting sleep, micro-sleeps can be observed.

According to a first simplified embodiment, said estimation rule comprises a comparison between the measured value of the driver monitoring parameter and a threshold value of this monitoring parameter.

For example, when the driver monitoring parameter is a closure frequency or period of the eyelids of the driver, the first estimation rule can comprise a comparison between the measured frequency or period value with a threshold frequency or period value. When the measured frequency or period value is greater than the threshold frequency or period value, the first estimation rule returns as output an unsatisfactory state of alertness of the driver, for example, indicating that they have lost alertness, are slightly distracted, highly distracted, or even falling asleep.

By way of an illustration, a slightly distracted state can be determined, for example, when the driver shifts their gaze from the road for a time interval ranging from 1.6 to 2 seconds and a highly distracted state can be determined, for example, when the driver shifts their gaze from the road for a time interval that is strictly greater than 2 seconds.

According to a second preferred embodiment, said estimation rule is determined by a learning algorithm adapted to establish a causality structure between the monitoring parameter and the state of alertness of the driver. This learning algorithm is initially trained using an initial predefined database comprising pairs of input variables comprising a monitoring parameter and a predefined associated state of alertness.

This initial database comprises data relating to various drivers, so that the initial performance levels of the estimation rule are satisfactory for most drivers.

The initial database is distinct from the database 200 personalized by the user.

The learning algorithm determines, for example, on the basis of this database, the probability of observing a state of alertness and preferably the state of distraction or of drowsiness provided by the driver, knowing that the monitoring parameter has said measured value.

The estimation rule returns as output, on the basis of the learning algorithm, the state of alertness, and particularly the most probable state of drowsiness or of distraction in accordance with the measured value of the driver monitoring parameter.

Preferably, regardless of the embodiment, a plurality of measured values of the monitoring parameter is measured and the state of alertness of the driver is estimated, and particularly the state of drowsiness or of distraction, in accordance with this plurality of measured values.

By way of a variation, the control unit can also receive data transmitted by other devices of the vehicle, such as data relating to GPS, acceleration/braking, activation of certain vehicle functions, such as, for example, the activation of the wireless connection of a mobile telephone to the vehicle.

Data indicating a driving trajectory that is inappropriate for the road can be taken into account in the estimation rule for determining the state of alertness of the driver. This data can include, for example, the lateral deviation of the vehicle in relation to the center of its path, the lateral stability of the vehicle, the frequency and the amplitude of micro-corrections exerted by the driver on the steering wheel. This data can also alternatively form the measured driver monitoring parameters.

The monitoring device 500 of the invention further comprises a device for emitting an alarm signal 400 (FIG. 1).

The emission of this alarm signal allows the driver to be notified of an insufficient state of alertness. The driver can then act to improve their state of alertness, for example, by taking a break in order to rest or by interrupting a telephone conversation.

The alarm signal emitted by the alarm signal emission device can be visual, audible and/or haptic.

The visual signal can involve turning on an indicator, blinking or a luminous flash emitted on the dashboard, the central rear-view mirror and/or the front side pillars of the vehicle.

The audible warning can be a recorded message or a sound that is optionally selected from a set of tones or music, optionally selected from a set of soundtracks.

The haptic signal can involve a vibration in the steering wheel, the seat belt and/or the driver seat.

The monitoring device according to the invention also comprises an interactive interface with the driver allowing them to select one or more alarm signal(s) adapted to be emitted by the emission device, from a predefined set of alarm signals.

Finally, the device for monitoring the driver according to the invention comprises a personalized database relating to the alarm signals, in which each selected alarm signal is recorded in correspondence with a driver identifier.

For example, the personalized database 200 relating to the alarm signals of the monitoring device according to the invention can allow driver files to be established, with each file comprising, for a given driver, the one or more alarm signal(s) selected by said driver.

The personalized database 200 relating to the alarm signals (and particularly the driver files) can be integrated, for example, in the previously defined control unit.

The driver can identify themselves, for example, by means of a dialogue box, as soon as they enter the motor vehicle.

By way of a variation, the relevant motor vehicle advantageously can further comprise a driver recognition system.

This recognition system automatically identifies the vehicle driver from a stored list of drivers. This recognition system is based, for example, on the facial recognition of the driver based on an image of the head of the driver, on processing biometric information (such as a fingerprint) or on the identification of a contact key used by the driver.

This recognition system can, for example, send the driver identifier to the control unit, which is then programmed to select the information from the personalized database 200 relative to the alarm signals in relation to this driver identifier. For example, the control unit can be programmed to select the driver file corresponding to the identifier transmitted by the recognition system.

Method

FIG. 1 schematically shows the steps of the monitoring method according to the invention.

The driver drives the vehicle as normal, without any particular restriction.

The monitoring method according to the invention is implemented while the vehicle is driven by the driver. This method comprises the following steps:

a) estimating 30 the state of alertness of said driver; and

b) emitting 40 at least one alarm signal intended to notify the driver of their state of alertness, in accordance with the estimated state of alertness of said driver.

It further comprises the following prior steps of personalizing the emission of the alarm signal:
selecting 10, by the driver, one or more alarm signal(s) adapted to be emitted during step b), from a predefined set of alarm signals;
storing 20 each selected alarm signal, in correspondence with a driver identifier, in said personalized database 200.

The estimated state of alertness of the driver advantageously belongs to a predefined set of possible states of alertness of the driver.

Preferably, estimating the state of alertness comprises estimating a state of drowsiness or a state of distraction of the driver, as previously defined.

Advantageously, during the selection step, the driver associates each selected alarm signal with one of the possible states of alertness of the driver and, during the recording step, the selected alarm signal is recorded in correspondence with the associated possible state of alertness.

Estimating the state of alertness advantageously comprises a step of measuring at least one driver monitoring parameter and a step of using at least one measured value of said at least one monitoring parameter measured to estimate a state of alertness of the driver in accordance with said estimation rule and the measured value of said monitoring parameter.

Such steps of measuring the monitoring parameter and of using said measurement of the monitoring parameter are, in a particularly advantageous manner, implemented by the measuring device and the control unit according to the previously described procedures.

In accordance with the state of alertness of the driver estimated by the control unit, said control unit may or may not control the emission of an alarm signal intended for the driver.

Said alarm signal is emitted when the device for estimating the state of alertness returns as output an unsatisfactory state of alertness, as previously defined. Advantageously, an alarm signal is emitted when at least one of the following states of distraction or drowsiness is detected: slight distraction (visual and/or audible), high distraction (visual and/or audible), loss of alertness or drowsiness.

According to the monitoring method of the invention, the emission of the alarm signal is personalized by the driver so that this emitted signal is better accepted by this driver. At least one of the following features of the alarm signal can be personalized by the driver:
the nature of the alarm signal, for example, visual, audible and/or haptic, as previously defined;
the intensity and/or the frequency of the alarm signal;
the potential number of repetitions of the alarm signal;
where necessary, the delay between two repetitions of said alarm signal.

During the recording step, each of these features is preferably recorded in said personalized database 200, in correspondence with the identifier of the carrier. By way of a variation, one or more feature(s) from these features is/are stored in said personalized database 200, in correspondence with this identifier of the carrier.

The number of repetitions of the alarm signal is an integer that can be zero.

Indeed, the alarm signal can be repeated once or twice, for example. In these conditions, the driver can decide whether or not to change the alarm signal upon each repetition.

For example, they can select a first visual signal, then an audible signal. They particularly can decide to increase the intensity of the alarm signal upon each repetition. When the alarm signal is repeated more than once, the driver can select various delays between each repetition of the alarm signal.

In the case of an audible or haptic signal, it is possible to provide for the driver to also select the audible frequency or the frequency of the vibrations emitted by the alarm signal emission device.

Preferably, when states of distraction or drowsiness are identified, the driver associates a different alarm signal according to the detected state of drowsiness or distraction.

In particular, they can assign a different alarm signal according to the detected state of distraction or drowsiness, in particular, depending on whether slight distraction, high distraction, loss of alertness or sleepiness is detected.

Optionally, various alarm signals can be combined. For example, for some states of alertness, the driver can select a combination of a plurality of alarm signals intended to be emitted simultaneously.

Furthermore, all the alarm signals may not necessarily be associated with all the states of alertness. More specifically, associating some alarm signals with some states of alertness can be prohibited.

For example, the driver can be excluded from associating a visual alarm signal with a state of drowsiness corresponding to sleepiness. By way of a variation, when the driver selects that a visual signal must be emitted when a state of drowsiness is detected, combining it with another alarm signal may be required.

The method of the invention can also exclude the possibility of associating an audible alarm signal with an audible distraction (slight or high) and a visual signal with a visual distraction (slight or high).

It also may be stipulated for a haptic alarm signal to be associated with any visual or audible alarm signal.

Furthermore, some features of the alarm signals can be provided by default for some states of alertness, without the driver having to modify them.

For example, the intensity of the alarm signal associated with the states of distraction and drowsiness associated with high distraction or sleepiness of the driver can be programmed to the maximum by default. Similarly, for these states of alertness, an audible alarm and/or a combination of an audible, visual and/or haptic alarm can be programmed by default, which is excluded from the personalization.

According to the method of the invention, each selected alarm signal is recorded in a personalized database 200 in correspondence with a driver identifier.

Advantageously, driver files containing all the selections of the driver relating to the personalizable features of the one or more alarm signal(s) are recorded in correspondence with the driver identifier.

Advantageously, in the method of the invention, when the driver enters the vehicle, they are automatically identified by the recognition device as previously described upon start-up of the vehicle, or they identify themselves. The personalized database 200, corresponding to the identified driver, is then recovered. In these conditions, when an unsatisfactory state of alertness is detected, the one or more alarm signal(s) corresponding to the selections of the identified driver are emitted.

In particular, the method of the invention allows, once the driver is identified:
during step a), the state of alertness of the driver to be estimated, preferably belonging to a predefined set of possible states of alertness, for example, belonging to the states of alertness as previously defined; and
during step b) of the method of the invention, emission, where necessary, of the alarm signal recorded in the personalized database 200 in correspondence with the identifier of the identified driver and for which the possible associated state of alertness corresponds to the state of alertness of the driver estimated in step a).

The monitoring method according to the invention can also comprise a step, when the driver has been identified and the corresponding personalized database 200 has been recovered, in which the driver is asked to modify their prior personalization selections of the one or more alarm signal(s).

Such an option is advantageous, for example, when the driver has previously selected one or more audible alarm(s) but is then accompanied by a passenger that they do not want to disturb. Thus, a visual or haptic alarm signal may be preferable and be selected for the current trip, so as not to disrupt the potential rest of the passenger.

Advantageously, the method of the invention allows the driver to test the various types of alarm signal, prior to the step of selecting the alarm signal. With the driver thus having knowingly selected their preferred type of alarm, the emission of the alarm signal when an unsatisfactory state of alertness of the driver is detected is better accepted.

The method for monitoring the driver according to the invention can also comprise a step, prior to the selection of one or more alarm signal(s) adapted to be emitted during step b) by the driver, of testing the effectiveness of each alarm signal.

Advantageously, in such an embodiment of the method of the invention, the alarm signal emission device emits a test alarm signal of predefined type and intensity.

Such a signal is preferably emitted, during a test phase, when the device for estimating the state of alertness has detected an unsatisfactory state of alertness. In these conditions, the test alarm signal is considered to be effective if a satisfactory state of alertness is detected after the emission of said test alarm signal or if the driver decides to stop driving within a predefined period.

Thus, preferably, the one or more alarm signal(s) proposed to the driver is/are previously tested and preselected for their effectiveness over a set of drivers.

By way of a variation, the one or more alarm signal(s) proposed to the driver is/are previously tested and preselected for their specific effectiveness over the driver of the vehicle.

For example, the test of the effectiveness of the alarm signal then advantageously allows a threshold intensity (visual, audible or haptic) of the alarm signal to be determined, below which the alarm signal is ineffective with respect to notifying the driver of their state of alertness. Preferably, this threshold intensity is stored in a memory for each type of alarm, preferably in correspondence with the driver identifier, so that the driver cannot select an intensity below this threshold intensity.

1. A method for monitoring the driver of a motor vehicle, comprising the following steps:
a) estimating a state of alertness of said driver; and
b) emitting at least one alarm signal intended to notify the driver of their state of alertness, in accordance with the estimated state of alertness of said driver;
personalizing the alarm signal by:
selecting, by the driver, said at least one alarm signal adapted to be emitted during step b), from a predefined set of alarm signals; and
recording each selected alarm signal in correspondence with a driver identifier in a personalized database.

2. The method for monitoring the driver of a motor vehicle as claimed in claim 1, wherein, during the selection step, the driver selects each alarm signal from one or more signal(s) of a visual, audible or haptic type, or from any one of their combinations.

3. The method for monitoring the driver of a motor vehicle as claimed in claim 1, wherein:
during the selection step, the driver makes a selection for a predefined number of repetitions of the emission of said alarm signal to be performed as well as, optionally, a delay between two repetitions of this alarm signal, and
during the recording step, the number of repetitions and the delay between two repetitions are recorded in said personalized database, in correspondence with the driver identifier.

4. The method for monitoring the driver of a motor vehicle as claimed in claim 1, wherein:
with said estimated state of alertness belonging to a predefined set of possible states of alertness, during the selection step, the driver associates each selected alarm signal with one of said possible states of alertness of the driver, and
during the recording step, the selected alarm signal is recorded in correspondence with the possible associated state of alertness.

5. The method for monitoring the driver of a motor vehicle as claimed in claim 1, wherein estimating the state of alertness comprises estimating a state of drowsiness or distraction.

6. The method for monitoring the driver of a motor vehicle as claimed in claim 5, wherein:
during step a), the state of drowsiness of the driver can be estimated as being an alert aware, loss of alertness or sleepiness state, and the state of distraction of the driver can be estimated as being a concentrated, slightly distracted or highly distracted state, and
during step b), said alarm signal is emitted when the state of drowsiness of the driver indicates that they have lost alertness or are in the process of falling asleep or when the state of distraction of the driver indicates that they are slightly or highly distracted.

7. The method for monitoring the driver of a motor vehicle as claimed in claim 5, wherein:
during the selection step, the driver associates the selected alarm signal with each possible state of distraction or drowsiness, and
during the recording step, said selected alarm signal is recorded in the personalized database in correspondence with the associated possible state of distraction or drowsiness.

8. The method for monitoring the driver of a motor vehicle as claimed in claim 1, wherein the identifier of the driver of the vehicle is also automatically determined and, during step b), the alarm signal is emitted that is recorded in the personalized database in correspondence with this identifier and corresponding to the state of alertness of the driver estimated in step a).

9. The method for monitoring the driver of a motor vehicle as claimed in claim 1, wherein the identifier of the driver of the vehicle is determined automatically, the personalized database associated with this identifier is recovered and the driver is asked to modify each selected alarm signal for the current trip.

10. The method for monitoring the driver of a motor vehicle as claimed in claim 1, wherein, prior to the selection step, the driver tests each alarm signal.

11. The method for monitoring the driver of a motor vehicle as claimed in claim 1, wherein a prior step is carried out of testing the effectiveness of each alarm signal adapted to be emitted to notify the driver of their state of alertness.

12. A device for monitoring the driver of a motor vehicle, comprising:
a device for estimating a state of alertness of the driver;
a device for emitting an alarm signal intended to notify the driver of their state of alertness, in accordance with the estimated state of alertness of the driver;
an interactive interface with the driver allowing them to select one or more alarm signal(s) adapted to be emitted by the emission device, from a predefined set of alarm signals;
a personalized database, in which each selected alarm signal is recorded in correspondence with a driver identifier.
